(19) United States
(12) Patent Application Publication
(10) Pub. No.: US 2024/0268885 A1
(43) Pub. Date: Aug. 15, 2024

(54) INTRAVASCULAR ATRIAL FIBRILLATION TREATMENT SYSTEM AND METHOD

(71) Applicant: CHAMBERTECH LTD., Kew, Richmond (GB)

(72) Inventor: Richard CHAMBERS, Kew (GB)

(73) Assignee: CHAMBERTECH LTD., Kew, Richmond (GB)

(21) Appl. No.: 18/569,082

(22) PCT Filed: Aug. 12, 2022
(86) PCT No.: PCT/US2022/040163

§ 371 Date: Dec. 11, 2023

Related U.S. Application Data
(60) Provisional application No. 63/260,234, filed on Aug. 13, 2021.

(51) Int. Cl. A61B 18/14; A61B 18/00

(52) U.S. Cl. CPC A61B 18/1492; A61B 2018/00369

(57) ABSTRACT

In accordance with the inventive concepts, provided are systems and methods for treating atrial arrhythmia. A system can comprise an elongate proximal shaft and a distal ablation probe, which is: (a) supported at a distal end of the elongate proximal shaft, (b) shaped, when unconstrained, so as to define at least first and second elongate ablating surfaces running alongside each other, and (c) configured to make, in an atrial wall of a heart, one or more ablation lesions that include at least first and second elongate continuous ablation lesion segments that are spaced apart and run alongside each other. The method uses the system to make the lesions.

### CROSS REFERENCE TO RELATED APPLICATIONS

The present application claims priority to U.S. Provisional Patent Application Ser. No. 63/260,234 filed Aug. 13, 2021, entitled “Intravascular Atrial Fibrillation Treatment,” which is hereby incorporated by reference in its entirety.

The present application, while not claiming priority to, may be related to Patent Cooperation Treaty (PCT) Application Serial No. PCT/US22/38464, entitled “Tissue Treatment System”, filed Jul. 27, 2022, which claimed priority to U.S. Provisional Patent Application Ser. No. 63/335,939, entitled “Tissue Treatment System”, filed Apr. 28, 2022 and to U.S. Provisional Patent Application Ser. No. 63/203,606, entitled “Tissue Treatment System”, filed Jul. 27, 2021, each of which is hereby incorporated by reference.

The present application, while not claiming priority to, may be related to Patent Cooperation Treaty (PCT) Application Serial No. PCT/US22/038461, entitled “Energy Delivery Systems with Lesion Index”, filed Jul. 27, 2022, which claims priority to U.S. Provisional Application Ser. No. 63/336,245, entitled “Energy Delivery Systems with Lesion Index”, filed Apr. 28, 2022 and U.S. Provisional Application Ser. No. 63/226,040, entitled “Energy Delivery Systems with Lesion Index”, filed Jul. 27, 2021, which is hereby incorporated by reference.

The present application, while not claiming priority to, may be related to US national stage filing of Patent Cooperation Treaty Application No. PCT/US2022/016722, entitled “Energy Delivery Systems with Ablation Index”, filed Feb. 17, 2022, which claims priority to U.S. Provisional Application Ser. No. 63/150,555, entitled “Energy Delivery Systems with Ablation Index”, filed Feb. 17, 2021, each of which is hereby incorporated by reference.

The present application, while not claiming priority to, may be related to U.S. application Ser. No. 16/335,893, entitled “Ablation System with Force Control”, filed Mar. 22, 2019, which is a 35 USC 371 national stage filing of Patent Cooperation Treaty Application No. PCT/US2017/056064, entitled “Ablation System with Force Control”, filed Oct. 11, 2017, which claims priority to U.S. Provisional Application Ser. No. 62/406,748, entitled “Ablation System with Force Control”, filed Oct. 11, 2016, and U.S. Provisional Application Ser. No. 62/504,139, entitled “Ablation System with Force Control”, filed May 10, 2017, each of which is hereby incorporated by reference.

The present application, while not claiming priority to, may be related to U.S. application Ser. No. 16/097,955, entitled “Cardiac Information Dynamic Display System and Method”, filed Oct. 31, 2018, which is a 35 USC 371 national stage filing of Patent Cooperation Treaty Application No. PCT/US2017/030915, entitled “Cardiac Information Dynamic Display System and Method”, filed May 3, 2017, which claims priority to US Provisional Application Ser. No. 62/331,351, entitled “Cardiac Information Dynamic Display System and Method”, filed May 3, 2016, each of which is hereby incorporated by reference.

The present application, while not claiming priority to, may be related to U.S. patent application Ser. No. 16/861,814, entitled “Catheter System and Methods of Medical Uses of Same, including Diagnostic and Treatment Uses for the Heart”, filed Apr. 29, 2020, which is a continuation of U.S. Pat. No. 10,667,753, entitled “Catheter System and Methods of Medical Uses of Same, Including Diagnostic and Treatment Uses for the Heart”, filed Jun. 19, 2018, which is a continuation of U.S. Pat. No. 10,004,459, entitled “Catheter System and Methods of Medical Uses of Same, Including Diagnostic and Treatment Uses for the Heart”, filed Feb. 20, 2015, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. patent application Ser. No. 16/242,810, entitled “Expandable Catheter Assembly with Flexible Printed Circuit Board (PCB) Electrical Pathways”, filed Jan. 8, 2019, which is a continuation of U.S. Pat. No. 10,201,311, entitled “Expandable Catheter Assembly with Flexible Printed Circuit Board (PCB) Electrical Pathways”, filed Jul. 23, 2015, which is a 35 USC 371 national stage filing of Patent Cooperation Treaty Application No. PCT/US2014/015261, entitled “Expandable Catheter Assembly with Flexible Printed Circuit Board (PCB) Electrical Pathways”, filed Feb. 7, 2014, which claims priority to U.S. Patent Provisional Application Ser. No. 61/762,363, entitled “Expandable Catheter Assembly with Flexible Printed Circuit Board (PCB) Electrical Pathways”, filed Feb. 8, 2013, each of which is hereby incorporated by reference.

The present application, while not claiming priority to, may be related to U.S. patent application Ser. No. 16/533,028, entitled “Method and Device for Determining and Presenting Surface Charge and Dipole Densities on Cardiac Walls”, filed Aug. 6, 2019, which is a continuation of U.S. Pat. No. 10,413,206, entitled “Method and Device for Determining and Presenting Surface Charge and Dipole Densities on Cardiac Walls”, filed Jun. 21, 2018, which is a continuation of U.S. Pat. No. 10,376,171, entitled “Method and Device for Determining and Presenting Surface Charge and Dipole Densities on Cardiac Walls”, filed Feb. 17, 2017, which is a continuation of U.S. Pat. No. 9,610,024, entitled “Method and Device for Determining and Presenting Surface Charge and Dipole Densities on Cardiac Walls”, filed Sep. 25, 2015, which is a continuation of U.S. Pat. No. 9,167,982, entitled “Method and Device for Determining and Presenting Surface Charge and Dipole Densities on Cardiac Walls”, filed Nov. 19, 2014, which is a continuation of U.S. Pat. No. 8,918,158, entitled “Method and Device for Determining and Presenting Surface Charge and Dipole Densities on Cardiac Walls”, filed Feb. 25, 2014, which is a continuation of U.S. Pat. No. 8,700,119, entitled “Method and Device for Determining and Presenting Surface Charge and Dipole Densities on Cardiac Walls”, filed Apr. 8, 2013, which is a continuation of U.S. Pat. No. 8,417,313, entitled “Method and Device for Determining and Presenting Surface Charge and Dipole Densities on Cardiac Walls”, filed Feb. 3, 2009, which is a 35 USC 371 national stage filing of PCT Application No. PCT/CH2007/000380, entitled “Method and Device for Determining and Presenting Surface Charge and Dipole Densities on Cardiac Walls”, filed Aug. 3, 2007, which claims priority to Swiss Patent Application No. 1251/06, filed Aug. 3, 2006, each of which is hereby incorporated by reference.

The present application, while not claiming priority to, may be related to U.S. Pat. No. 11,116,438, entitled “Device and Method for the Geometric Determination of Electrical Dipole Densities on the Cardiac Wall”, filed Sep. 12, 2019, which is a continuation of U.S. Pat. No. 10,463,267, entitled “Device and Method for the Geometric Determination of Electrical Dipole Densities on the Cardiac Wall”, filed Jan. 29, 2018, which is a continuation of U.S. Pat. No. 9,913,589, entitled “Device and Method for the Geometric Determination of Electrical Dipole Densities on the Cardiac Wall”, filed Oct. 25, 2016, which is a continuation of U.S. Pat. No. 9,504,395, entitled “Device and Method for the Geometric Determination of Electrical Dipole Densities on the Cardiac Wall”, filed Oct. 19, 2015, which is a continuation of U.S. Pat. No. 9,192,318, entitled “Device and Method for the Geometric Determination of Electrical Dipole Densities on the Cardiac Wall”, filed Jul. 19, 2013, which is a continuation of U.S. Pat. No. 8,512,255, entitled “Device and Method for the Geometric Determination of Electrical Dipole Densities on the Cardiac Wall”, filed Jul. 16, 2010, which is a 35 USC 371 national stage application of Patent Cooperation Treaty Application No. PCT/IB2009/000071, filed Jan. 16, 2009, entitled “A Device and Method for the Geometric Determination of Electrical Dipole Densities on the Cardiac Wall”, which claimed priority to Swiss Patent Application 00068/08 filed Jan. 17, 2008, each of which is hereby incorporated by reference.

The present application, while not claiming priority to, may be related to U.S. patent application Ser. No. 17/673,995, entitled “Device and Method for the Geometric Determination of Electrical Dipole Densities on the Cardiac Wall”, filed Feb. 17, 2022, which is a continuation of U.S. Pat. No. 11,278,209, entitled “Device and Method for the Geometric Determination of Electrical Dipole Densities on the Cardiac Wall”, filed Apr. 19, 2019, which is a continuation of U.S. Pat. No. 10,314,497, entitled “Device and Method for the Geometric Determination of Electrical Dipole Densities on the Cardiac Wall”, filed Mar. 20, 2018, which is a continuation of U.S. Pat. No. 9,968,268, entitled “Device and Method for the Geometric Determination of Electrical Dipole Densities on the Cardiac Wall”, filed Aug. 8, 2017, which is a continuation of U.S. Pat. No. 9,757,044, entitled “Device and Method for the Geometric Determination of Electrical Dipole Densities on the Cardiac Wall”, filed Sep. 6, 2013, which is a 35 USC 371 national stage filing of Patent Cooperation Treaty Application No. PCT/US2012/028593, entitled “Device and Method for the Geometric Determination of Electrical Dipole Densities on the Cardiac Wall”, filed Mar. 9, 2012, which claimed priority to U.S. Patent Provisional Application Ser. No. 61/451,357, filed Mar. 10, 2011, each of which is hereby incorporated by reference.

The present application, while not claiming priority to, may be related to US Design Patent No. 29/681,827, entitled “Set of Transducer-Electrode Pairs for a Catheter”, filed Feb. 28, 2019, which is a division of US Design Patent No. D851,774, entitled “Set of Transducer-Electrode Pairs for a Catheter”, filed Feb. 6, 2017, which is a division of US Design Patent No. D782,686, entitled “Transducer-Electrode Pair for a Catheter”, filed Dec. 2, 2013, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. patent application Ser. No. 16/111,538, entitled “Gas-Elimination Patient Access Device”, filed Aug. 24, 2018, which is a continuation of U.S. Pat. No. 10,071,227, entitled “Gas-Elimination Patient Access Device”, filed Jul. 14, 2016, which is a 35 USC 371 national stage filing of Patent Cooperation Treaty Application No. PCT/US2015/011312, entitled “Gas-Elimination Patient Access Device”, filed Jan. 14, 2015, which claims priority to U.S. Patent Provisional Application Ser. No. 61/928,704, entitled “Gas-Elimination Patient Access Device”, filed Jan. 17, 2014, which is hereby incorporated by reference.

The present application, while not claiming priority to, may be related to U.S. patent application Ser. No. 17/578,522, entitled “Cardiac Analysis User Interface System and Method”, filed Jan. 19, 2022, which is a continuation of U.S. Pat. No. 11,278,231, entitled “Cardiac Analysis User Interface System and Method”, filed Sep. 23, 2016, which is a 35 USC 371 national stage filing of Patent Cooperation Treaty Application No. PCT/US2015/022187, entitled “Cardiac Analysis User Interface System and Method”, filed Mar. 24, 2015, which claims priority to U.S. Patent Provisional Application Ser. No. 61/970,027, entitled “Cardiac Analysis User Interface System and Method”, filed Mar. 25, 2014, which is hereby incorporated by reference.

The present application, while not claiming priority to, may be related to U.S. patent application Ser. No. 17/063,901, entitled “Devices and Methods for Determination of Electrical Dipole Densities on a Cardiac Surface”, filed Oct. 6, 2020, which is a continuation of U.S. Pat. No. 10,828,011, entitled “Devices and Methods for Determination of Electrical Dipole Densities on a Cardiac Surface”, filed Mar. 2, 2016, which is a 35 USC 371 national stage filing of Patent Cooperation Treaty Application No. PCT/US2014/054942, entitled “Devices and Methods for Determination of Electrical Dipole Densities on a Cardiac Surface”, filed Sep. 10, 2014, which claims priority to U.S. Patent Provisional Application Ser. No. 61/877,617, entitled “Devices and Methods for Determination of Electrical Dipole Densities on a Cardiac Surface”, filed Sep. 13, 2013, which is hereby incorporated by reference.

The present application, while not claiming priority to, may be related to U.S. patent application Ser. No. 16/849,045, entitled “Localization System and Method Useful in the Acquisition and Analysis of Cardiac Information”, filed Apr. 15, 2020, which is a continuation of U.S. Pat. No. 10,653,318, entitled “Localization System and Method Useful in the Acquisition and Analysis of Cardiac Information”, filed Oct. 26, 2017, which is a 35 USC 371 national stage filing of Patent Cooperation Treaty Application No. PCT/US2016/032420, entitled “Localization System and Method Useful in the Acquisition and Analysis of Cardiac Information”, filed May 13, 2016, which claims priority to U.S. Patent Provisional Application Ser. No. 62/161,213, entitled “Localization System and Method Useful in the Acquisition and Analysis of Cardiac Information”, filed May 13, 2015, which is hereby incorporated by reference.

The present application, while not claiming priority to, may be related to U.S. patent application Ser. No. 15/569,231, entitled “Cardiac Virtualization Test Tank and Testing System and Method”, filed Oct. 25, 2017, which is a 35 USC 371 national stage filing of Patent Cooperation Treaty Application No. PCT/US2016/031823, filed May 11, 2016, which claims priority to U.S. Patent Provisional Application Ser. No. 62/160,501, entitled “Cardiac Virtualization Test Tank and Testing System and Method”, filed May 12, 2015, which is hereby incorporated by reference.

The present application, while not claiming priority to, may be related to U.S. patent application Ser. No. 17/735,285, entitled “Ultrasound Sequencing System and Method”, filed May 3, 2022, which is a continuation of to U.S. patent application Ser. No. 15/569,185, entitled “Ultrasound Sequencing System and Method”, filed Oct. 25, 2017, which is a 35 USC 371 national stage filing of Patent Cooperation Treaty Application No. PCT/US2016/032017, filed May 12, 2016, which claims priority to U.S. Patent Provisional Application Ser. No. 62/160,529, entitled “Ultrasound Sequencing System and Method”, filed May 12, 2015, which is hereby incorporated by reference.

The present application, while not claiming priority to, may be related to U.S. patent application Ser. No. 17/858,174, entitled “Cardiac Mapping System with Efficiency Algorithm”, filed Jul. 6, 2022, which is a Continuation Application of U.S. patent application Ser. No. 16/097,959, entitled “Cardiac Mapping System with Efficiency Algorithm”, filed Oct. 31, 2018, which is a 35 USC 371 national stage filing of Patent Cooperation Treaty Application No. PCT/US2017/030922, entitled “Cardiac Mapping System with Efficiency Algorithm”, filed May 3, 2017, which claims priority to U.S. Patent Provisional Application Ser. No. 62/413,104, entitled “Cardiac Mapping System with Efficiency Algorithm”, filed Oct. 26, 2016, and U.S. Patent Provisional Application Ser. No. 62/331,364, entitled “Cardiac Mapping System with Efficiency Algorithm”, filed May 3, 2016, each of which is hereby incorporated by reference.

The present application, while not claiming priority to, may be related to U.S. patent application Ser. No. 16/961,809, entitled “System for Identifying Cardiac Conduction Patterns”, filed Jul. 13, 2020, which is a 35 USC 371 national stage filing of Patent Cooperation Treaty Application No. PCT/US2019/014498, entitled “System for Identifying Cardiac Conduction Patterns”, filed Jan. 22, 2019, which claims priority to U.S. Patent Provisional Application Ser. No. 62/619,897, entitled “System for Recognizing Cardiac Conduction Patterns”, filed Jan. 21, 2018, and U.S. Patent Provisional Application Ser. No. 62/668,647, entitled “System for Identifying Cardiac Conduction Patterns”, filed May 8, 2018, each of which is hereby incorporated by reference.

The present application, while not claiming priority to, may be related to U.S. patent application Ser. No. 17/048,151, entitled “Cardiac Information Processing System”, filed Oct. 16, 2020, which is a 35 USC 371 national stage filing of Patent Cooperation Treaty Application No. PCT/US2019/031131, entitled “Cardiac Information Processing System”, filed May 7, 2019, which claims priority to U.S. Provisional Application Ser. No. 62/668,659, entitled “Cardiac Information Processing System”, filed May 8, 2018, and U.S. Patent Provisional Application Ser. No. 62/811,735, entitled “Cardiac Information Processing System”, filed Feb. 28, 2019, each of which is hereby incorporated by reference.

The present application, while not claiming priority to, may be related to Patent Cooperation Treaty Application No. PCT/US2019/060433, entitled “Systems and Methods for Calculating Patient Information”, filed Nov. 8, 2019, which claims priority to U.S. Provisional Application Ser. No. 62/757,961, entitled “Systems and Methods for Calculating Patient Information”, filed Nov. 9, 2018, each of which is hereby incorporated by reference.

The present application, while not claiming priority to, may be related to U.S. patent application Ser. No. 17/601,661, entitled “System for Creating a Composite Map”, filed Oct. 5, 2021, which is a 35 USC 371 national stage filing of Patent Cooperation Treaty Application No. PCT/US2020/028779, entitled “System for Creating a Composite Map”, filed Apr. 17, 2020, which claims priority to U.S. Provisional Application Ser. No. 62/835,538, entitled “System for Creating a Composite Map”, filed Apr. 18, 2019, and U.S. Provisional Application Ser. No. 62/925,030, entitled “System for Creating a Composite Map”, filed Oct. 23, 2019, each of which is hereby incorporated by reference.

The present application, while not claiming priority to, may be related to U.S. patent application Ser. No. 17/613,249, entitled “Systems And Methods For Performing Localization Within A Body”, filed Nov. 22, 2021, which is a 35 USC 371 national stage filing of Patent Cooperation Treaty Application No. PCT/US2020/036110, entitled “Systems and Methods for Performing Localization Within a Body”, filed Jun. 4, 2020, which claims priority to U.S. Provisional Application Ser. No. 62/857,055, entitled “Systems and Methods for Performing Localization Within a Body”, filed Jun. 4, 2019, each of which is hereby incorporated by reference.

The present application, while not claiming priority to, may be related to U.S. patent application Ser. No. 17/777,104, entitled “Tissue Treatment Systems, Devices, and Methods”, filed May 16, 2022, which is a 35 USC 371 national stage filing of Patent Cooperation Treaty Application No. PCT/US2020/061458, entitled “Tissue Treatment Systems, Devices, and Methods”, filed Nov. 20, 2020, which claims priority to U.S. Provisional Application Ser. No. 62/939,412, entitled “Tissue Treatment Systems, Devices, and Methods”, filed Nov. 22, 2019, and U.S. Provisional Application Ser. No. 63/075,280, entitled “Tissue Treatment Systems, Devices, and Methods”, filed Sep. 7, 2020, each of which is hereby incorporated by reference.

### FIELD OF THE APPLICATION

The present invention relates generally to transcatheter surgical methods, and specifically to transcatheter surgical methods for treating atrial arrhythmias.

### BACKGROUND OF THE APPLICATION

It is estimated that patients suffering from persistent atrial fibrillation (AF) who undergo catheter ablation have a 50% first procedure success rate. It is generally believed that in order to improve this success rate, pulmonary vein isolation must be supplemented with ablation of areas of the left atrium in addition to the pulmonary veins.

The surgical ablation Cox-Maze procedure is a purely anatomical approach of creating surgical lines of scar/electrical block between electrically inert boundaries. This highly invasive procedure, usually via a thoracotomy approach, yields very high chronic procedural success rates (typically, greater than 85%), but carries a substantially greater risk of major procedure related complications than transcatheter (e.g., transvenous) ablation. Surgical ablation is also a significantly more expensive procedure, and requires longer recover time in the hospital.

Performing the Cox-Maze procedure using catheter ablation techniques requires exceptional skill levels. When successful, the catheter ablation has significantly better efficacy than pulmonary vein isolation alone, but results vary substantially based on the skill level of the operator.

Linear lesion sets remain one of the most challenging methodologies for catheter ablation, and are widely avoided due to the propensity for atrial tachycardias. Atrial tachycardias may continue to occur because of either a non-continuous lesion set(s) and/or tissue healing. Linear lesion sets are generally required to connect between two electrically inert anatomical boundaries, i.e., peripheral regions of the cardiac tissue in which the cardiac tissue transitions from myocytes to non-conductive tissue. However, not all linear lesion sets are anchored at either or both ends.

### SUMMARY OF THE APPLICATION

In accordance with aspects of the inventive concepts, an ablation catheter is provided for treating atrial arrhythmias, such as atrial fibrillation or atrial flutter. The ablation catheter comprises an elongate proximal shaft and a distal ablation probe, supported at a distal end of the elongate proximal shaft. The distal ablation probe is shaped, when unconstrained, so as to define at least first and second elongate ablating surfaces running alongside each other, and is configured to make, in an atrial wall of a heart, one or more ablation lesions that include at least first and second elongate continuous ablation lesion segments. The distal ablation probe is advanced to an atrium in a transcatheter (e.g., transvenous) procedure, and the first and the second elongate ablating surfaces are used to make the first and second elongate continuous ablation lesion segments, respectively.

Various embodiments of an ablation catheter in accordance with aspects of the inventive concepts can provide an easy-to-use solution that closely imitates surgical procedures, such as the Cox-Maze procedure, while reducing the required operator skill and resulting intra-operator variability. Embodiments, of the ablation catheter generally improve the acute and chronic success rates of ablation procedures by simplifying the creation of generally parallel double contiguous linear elongate lesion formations. By contrast, the simpler creation of single elongate lesions often does not provide long-term treatment of the atrial arrhythmia, because healthy tissue often bridges, i.e., grows across the single lesion.

There is therefore provided, in accordance with aspects of the inventive concepts, an ablation catheter useful to treat atrial arrhythmia, including: an elongate proximal shaft; and a distal ablation probe, which is (a) supported at a distal end of the elongate proximal shaft, (b) shaped, when unconstrained, so as to define at least first and second elongate ablating surfaces running alongside each other, and (c) configured to make, in an atrial wall of a heart, one or more ablation lesions that include at least first and second elongate continuous ablation lesion segments that are spaced apart and run alongside each other.

In some embodiments, the first and the second elongate ablating surfaces include first and second elongate continuous ablating surfaces, respectively.

In some embodiments: the first elongate ablating surface includes a first elongate discontinuous ablating surface, configured to make the first elongate continuous ablation lesion, and the second elongate ablating surface includes a second elongate continuous ablating surface, configured to make the second elongate continuous ablation lesion.

In some embodiments, the first and the second elongate ablating surfaces include first and second elongate discontinuous ablating surfaces, respectively.

In some embodiments, the distal ablation probe, when unconstrained, has greatest major and minor dimensions perpendicular to each other, the greatest major dimension at least 3 times the greatest minor dimension.

In some embodiments, the greatest major dimension is at least 4 times the greatest minor dimension.

In some embodiments, the distal ablation probe, when unconstrained, has a greatest dimension of between 4 and 10 cm.

In some embodiments, the first and the second elongate ablating surfaces are coplanar when the distal ablation probe is unconstrained.

In some embodiments, the first and the second elongate ablating surfaces are parallel to each other when the distal ablation probe is unconstrained.

In some embodiments, the first and the second elongate ablating surfaces run alongside each other for an ablation-surface length of between 4 and 8 cm when the distal ablation probe is unconstrained.

In some embodiments, a closest distance between the first and the second elongate ablating surfaces is between 5 and 20 mm when the distal ablation probe is unconstrained.

In some embodiments, the distal ablation probe includes one or more sensing electrodes.

In some embodiments, a distance between the first and the second elongate ablating surfaces does not vary along the first and the second elongate ablating surfaces when the distal ablation probe is unconstrained.

In some embodiments, a distance between the first and the second elongate ablating surfaces varies along the first and the second elongate ablating surfaces when the distal ablation probe is unconstrained.

In some embodiments, the first and the second elongate ablating surfaces are straight when the distal ablation probe is unconstrained.

In some embodiments, the first and the second elongate ablating surfaces are curved when the distal ablation probe is unconstrained.

In some embodiments, when the distal ablation probe is unconstrained, the first and the second elongate ablating surfaces have respective radii of curvature, each of which is between 0.2 and 1.2 cm.

In some embodiments, the distal ablation probe includes an elongate distal shaft that is shaped so as to define the first and the second elongate ablating surfaces when the distal ablation probe is unconstrained.

In some embodiments, when the distal ablation probe is unconstrained, the elongate distal shaft has greatest major and minor dimensions perpendicular to each other, and the greatest major dimension equals at least 3 times the greatest minor dimension.

In some embodiments, the greatest major dimension is at least 4 times the greatest minor dimension.

In some embodiments: a proximal end of the elongate distal shaft is supported at the distal end of the elongate proximal shaft, and when the distal ablation probe is unconstrained, a proximal portion of the elongate distal shaft forms an angle with a central longitudinal axis of the elongate proximal shaft that passes through the distal end of the elongate proximal shaft, the angle between 45 and 90 degrees, such as between 60 and 90 degrees.

In some embodiments: the first elongate ablating surface includes a first elongate discontinuous ablating surface, configured to make the first elongate continuous ablation lesion, and the second elongate ablating surface includes a second elongate continuous ablating surface, configured to make the second elongate continuous ablation lesion.

In some embodiments, a distal end of the elongate distal shaft is located along the first elongate ablating surface when the distal ablation probe is unconstrained.

In some embodiments, a distal end of the elongate distal shaft physically touches a proximal end of the elongate distal shaft when the distal ablation probe is unconstrained.

In some embodiments, when the distal ablation probe is unconstrained, an inner perimeter of the elongate distal shaft surrounds an area of between 2 and 16 cm2.

In some embodiments, the elongate distal shaft is shaped so as to define two curved connecting end portions that connect the first and the second elongate ablating surfaces when the distal ablation probe is unconstrained.

In some embodiments, the first and the second elongate ablating surfaces are curved and the elongate distal shaft is ovaloid when the distal ablation probe is unconstrained.

In some embodiments, the elongate distal shaft is stadium-shaped when the distal ablation probe is unconstrained.

In some embodiments, the ablation system further including an intravascular delivery sheath, in which the ablation catheter is removably disposed for delivery such that the elongate distal shaft is constrained by the intravascular delivery sheath, such that the first and the second elongate ablating surfaces are disposed at respective, non-longitudinally-overlapping locations along the intravascular delivery sheath.

In some embodiments, the distal ablation probe includes a shape memory material that causes the elongate distal shaft to define the first and the second ablating surfaces running alongside each other when the elongate distal shaft is unconstrained.

In some embodiments, when the distal ablation probe is unconstrained: a proximal end of the elongate distal shaft is supported at the distal end of the elongate proximal shaft, the first elongate ablating surfaces includes the proximal end of the elongate distal shaft, the proximal end is located at a location along the first elongate ablating surface at a distance from an endpoint of the first elongate ablating surface, the distance equal to between 40% and 60% of a length of the first elongate ablating surface.

In some embodiments, when the distal ablation probe is unconstrained, a best-fit plane defined by the first and the second elongate ablating surfaces forms an angle with a central longitudinal axis of the elongate proximal shaft that passes through the distal end of the elongate proximal shaft, the angle between 45 and 90 degrees, such as between 60 and 90 degrees.

In some embodiments, the first and the second elongate ablating surfaces are configured to apply cryoablation.

In some embodiments: the distal ablation probe includes an elongate distal shaft that is shaped so as to define the first and the second elongate ablating surfaces when the distal ablation probe is unconstrained, the distal elongate shaft includes inner and outer tubes, the inner tube is shaped so as to define a first lumen, the inner and the outer tubes together define a second lumen between an outer surface of the inner tube and an inner surface of the outer tube, and the first and the second lumens are in fluid communication with each other near a distal end of the distal elongate shaft.

In some embodiments, the ablation system further including a source of cryogenic fluid coupled in fluid communication with the first and the second lumens.

In some embodiments, the first and the second elongate ablating surfaces include respective sets of one or more ablation electrodes.

In accordance with another aspect of the inventive concepts, provided is a method for treating atrial arrhythmia including: advancing, in a transcatheter procedure, into an atrium of a heart, a distal ablation probe that is supported at a distal end of an elongate proximal shaft of an ablation catheter; deploying the distal ablation probe in the atrium such that the distal ablation probe is shaped so as to define at least first and second elongate ablating surfaces running alongside each other; and using the distal ablation probe, making, in an atrial wall, one or more ablation lesions that include at least first and second elongate continuous ablation lesion segments that are spaced apart and run alongside each other.

In some embodiments, making the one or more ablation lesions includes making the first and the second elongate continuous ablation lesion segments at respective locations in the atrial wall that connect electrically inert boundaries of the atrial wall.

In some embodiments, making the first and the second elongate continuous ablation lesion segments includes making the first and the second elongate continuous ablation lesion segments generally extending between: an orifice of a left superior pulmonary vein (LSPV) and an orifice of a right superior pulmonary vein (RSPV), an RSPV and a mitral valve (MV), a right inferior pulmonary vein (RIPV) and an MV, a left inferior pulmonary vein (LIPV) and an RIPV, an LSPV and an RIPV, or an RSPV and an LIPV.

In some embodiments, the method does not include using the distal ablation probe to perform pulmonary vein isolation.

In some embodiments, advancing the distal ablation probe while the ablation catheter is removably disposed for delivery in an intravascular delivery sheath, such that the elongate distal shaft is constrained by the intravascular delivery sheath, such that the first and the second elongate ablating surfaces are disposed at respective, non-longitudinally-overlapping locations along the intravascular delivery sheath.

In some embodiments, the first and the second elongate ablating surfaces include first and second elongate continuous ablating surfaces, respectively.

In some embodiments: the first elongate ablating surface includes a first elongate discontinuous ablating surface, configured to make the first elongate continuous ablation lesion, and the second elongate ablating surface includes a second elongate continuous ablating surface, configured to make the second elongate continuous ablation lesion.

In some embodiments, the first and the second elongate ablating surfaces include first and second elongate discontinuous ablating surfaces, respectively.

In some embodiments, the distal ablation probe, when unconstrained, has greatest major and minor dimensions perpendicular to each other, the greatest major dimension at least 3 times the greatest minor dimension.

In some embodiments, the greatest major dimension is at least 4 times the greatest minor dimension.

In some embodiments, the distal ablation probe, when unconstrained, has a greatest dimension of between 4 and 10 cm.

In some embodiments, the first and the second elongate ablating surfaces are coplanar when the distal ablation probe is unconstrained.

In some embodiments, the first and the second elongate ablating surfaces are parallel to each other when the distal ablation probe is unconstrained.

In some embodiments, the first and the second elongate ablating surfaces are straight when the distal ablation probe is unconstrained.

In some embodiments, the first and the second elongate ablating surfaces are curved when the distal ablation probe is unconstrained.

In some embodiments, when the distal ablation probe is unconstrained, the first and the second elongate ablating surfaces have respective radii of curvature, each of which is between 0.2 and 1.2 cm.

In some embodiments, the distal ablation probe includes an elongate distal shaft that is shaped so as to define the first and the second elongate ablating surfaces when the distal ablation probe is unconstrained.

In some embodiments, when the distal ablation probe is unconstrained, the elongate distal shaft has greatest major and minor dimensions perpendicular to each other, and the greatest major dimension equals at least 3 times the greatest minor dimension.

In some embodiments, the greatest major dimension is at least 4 times the greatest minor dimension.

In some embodiments: a proximal end of the elongate distal shaft is supported at the distal end of the elongate proximal shaft, and when the distal ablation probe is unconstrained, a proximal portion of the elongate distal shaft forms an angle with a central longitudinal axis of the elongate proximal shaft that passes through the distal end of the elongate proximal shaft, the angle between 45 and 90 degrees, such as between 60 and 90 degrees.

In some embodiments: the first elongate ablating surface includes a first elongate discontinuous ablating surface, configured to make the first elongate continuous ablation lesion, and the second elongate ablating surface includes a second elongate continuous ablating surface, configured to make the second elongate continuous ablation lesion.

In some embodiments, a distal end of the elongate distal shaft is located along the first elongate ablating surface when the distal ablation probe is unconstrained.

In some embodiments, a distal end of the elongate distal shaft physically touches a proximal end of the elongate distal shaft when the distal ablation probe is unconstrained.

In some embodiments, when the distal ablation probe is unconstrained, an inner perimeter of the elongate distal shaft surrounds an area of between 2 and 16 cm2.

In some embodiments, the elongate distal shaft is shaped so as to define two curved connecting end portions that connect the first and the second elongate ablating surfaces when the distal ablation probe is unconstrained.

In some embodiments, the first and the second elongate ablating surfaces are curved and the elongate distal shaft is ovaloid when the distal ablation probe is unconstrained.

In some embodiments, the elongate distal shaft is stadium-shaped when the distal ablation probe is unconstrained.

In some embodiments, the distal ablation probe includes a shape memory material that causes the elongate distal shaft to define the first and the second ablating surfaces running alongside each other when the elongate distal shaft is unconstrained.

In some embodiments, when the distal ablation probe is unconstrained: a proximal end of the elongate distal shaft is supported at the distal end of the elongate proximal shaft, the first elongate ablating surfaces includes the proximal end of the elongate distal shaft, the proximal end is located at a location along the first elongate ablating surface at a distance from an endpoint of the first elongate ablating surface, the distance equal to between 40% and 60% of a length of the first elongate ablating surface.

In some embodiments, when the distal ablation probe is unconstrained, a best-fit plane defined by the first and the second elongate ablating surfaces forms an angle with a central longitudinal axis of the elongate proximal shaft that passes through the distal end of the elongate proximal shaft, the angle between 45 and 90 degrees, such as between 60 and 90 degrees.

In some embodiments, the first and the second elongate ablating surfaces run alongside each other for an ablation-surface length of between 4 and 8 cm when the distal ablation probe is unconstrained.

In some embodiments, a closest distance between the first and the second elongate ablating surfaces is between 5 and 20 mm when the distal ablation probe is unconstrained.

In some embodiments, a distance between the first and the second elongate ablating surfaces does not vary along the first and the second elongate ablating surfaces when the distal ablation probe is unconstrained.

In some embodiments, a distance between the first and the second elongate ablating surfaces varies along the first and the second elongate ablating surfaces when the distal ablation probe is unconstrained.

In some embodiments, the first and the second elongate ablating surfaces are configured to apply cryoablation.

In some embodiments: the distal ablation probe includes an elongate distal shaft that is shaped so as to define the first and the second elongate ablating surfaces when the distal ablation probe is unconstrained, the distal elongate shaft includes inner and outer tubes, the inner tube is shaped so as to define a first lumen, the inner and the outer tubes together define a second lumen between an outer surface of the inner tube and an inner surface of the outer tube, and the first and the second lumens are in fluid communication with each other near a distal end of the distal elongate shaft.

In some embodiments, the method further includes coupling a source of cryogenic fluid in fluid communication with the first and the second lumens.

In some embodiments, the first and the second elongate ablating surfaces include respective sets of one or more ablation electrodes.

In some embodiments, the distal ablation probe includes one or more sensing electrodes.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 is a schematic illustration of an ablation system useful to treat atrial arrhythmias, in accordance with aspects of the inventive concepts;

FIGS. 2A and 2B are schematic illustrations of an ablation catheter of the ablation system, from distal and side views, respectively, in accordance with aspects of the inventive concepts;

FIGS. 3A and 3B are cross-sectional views of an elongate distal shaft of a distal ablation probe of the ablation catheter of FIGS. 1 and 2A-B taken along lines IIIA-IIIA and IIB-IIIB, respectively, of FIG. 2A, in accordance with aspects of the inventive concepts;

FIGS. 4A-F are schematic illustrations of a method for treating atrial arrhythmia, in accordance with aspects of the inventive concepts;

FIG. 5 is a schematic illustration of another ablation system for treating atrial arrhythmia, in accordance with aspects of the inventive concepts; and

FIG. 6 is a schematic illustration of yet another ablation system for treating atrial arrhythmia, in accordance with aspects of the inventive concepts.

### DETAILED DESCRIPTION OF APPLICATIONS

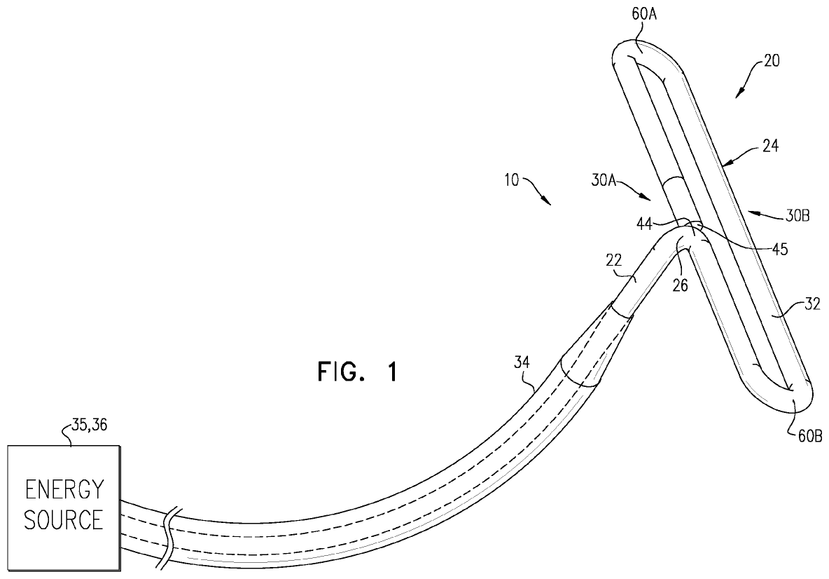

Reference is made to FIG. 1, which is a schematic illustration of an ablation system 10 for treating atrial arrhythmias, such as atrial fibrillation or atrial flutter, in accordance with an application of the present invention.

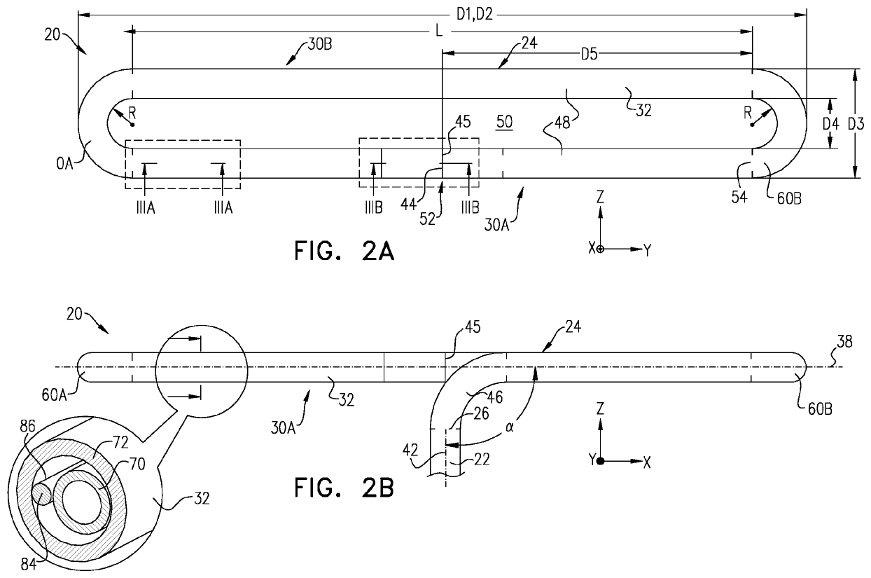

Reference is also made to FIGS. 2A and 2B, which are schematic illustrations of an ablation catheter 20 of ablation system 10, from distal and side views, respectively, in accordance with an application of the present invention.

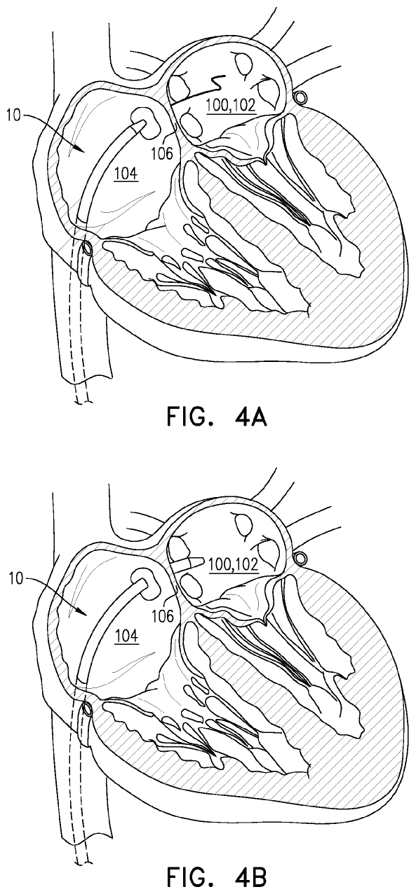

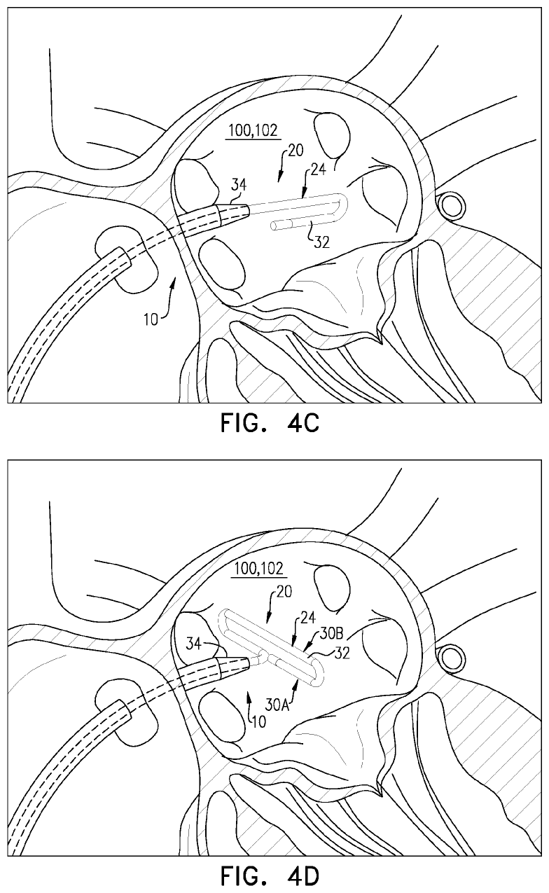

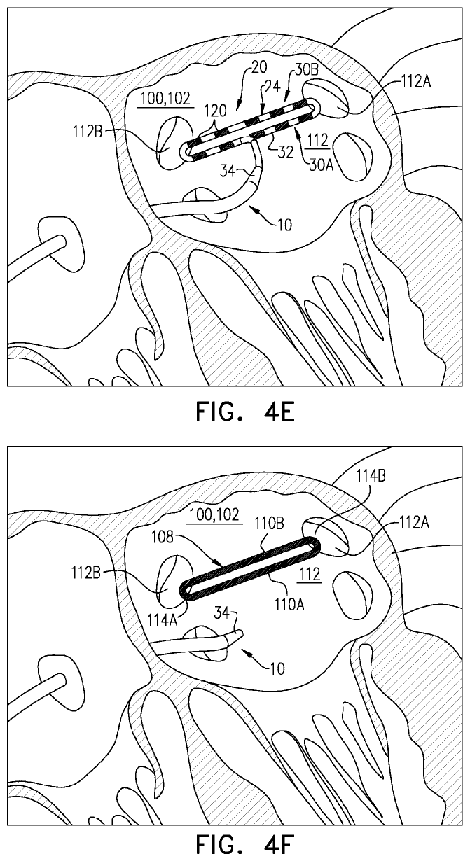

Ablation catheter 20 comprises an elongate proximal shaft 22 and a distal ablation probe 24, supported at a distal end 26 of elongate proximal shaft 22. Distal ablation probe 24 is shaped, when unconstrained (by intravascular delivery sheath 34, described hereinbelow, or by the subject's anatomy, or otherwise), so as to define at least first and second elongate ablating surfaces 30A and 30B running alongside each other. Distal ablation probe 24 is configured to make, in an atrial wall of a heart, one or more ablation lesions that include at least first and second elongate continuous ablation lesion segments that are spaced apart and run alongside each other. First and second elongate ablating surfaces 30A and 30B together define a transmissive region for applying ablation to make the first and the second spaced-apart elongate ablation lesions in the atrial wall, such as described hereinbelow with reference to FIGS. 4E-F.

For some applications, distal ablation probe 24 comprises an elongate distal shaft 32 that is shaped so as to define first and second elongate ablating surfaces 30A and 30B when distal ablation probe 24 is unconstrained.

For other applications, the distal ablation probe comprises, instead of the elongate distal shaft, a generally flat surface on which the first and the second elongate ablating surfaces are disposed; for example, the first and the second elongate ablating surfaces may comprise respective elongate electrode contact surfaces (configuration not shown). For example, the generally flat surface may be provided by a paddle-shaped element (configuration not shown).

Reference is again made to FIG. 1. Ablation system 10 further comprises an intravascular (typically, transvenous) delivery sheath 34, in which ablation catheter 20 is removably disposed for delivery. For some applications in which distal ablation probe 24 comprises elongate distal shaft 32, ablation catheter 20 is removably disposed in delivery sheath 34, such that elongate distal shaft 32 is constrained by delivery sheath 34 and, typically, first and second elongate ablating surfaces 30A and 30B are disposed at respective, non-longitudinally-overlapping locations along delivery sheath 34. Distal ablation probe 24 (e.g., elongate distal shaft 32) is sufficiently flexible to allow it to assume tortuous geometric formations as it is advanced within delivery sheath 34 around acute bends in the vasculature.

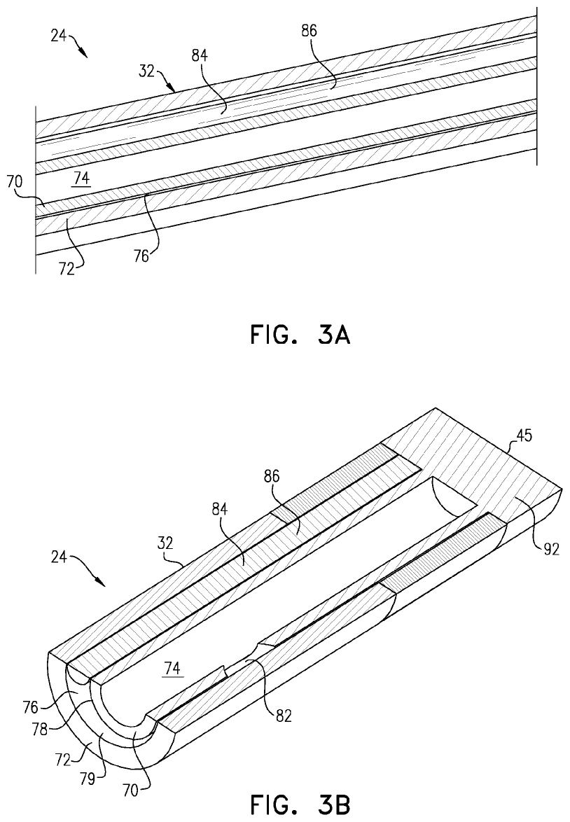

For some applications, ablation system 10 further comprises an energy source 35, which may be a thermal or non-thermal energy source. For example, energy source 35 may comprise a source 36 of cryogenic fluid coupled (via respective lumens defined by proximal shaft 22) in fluid communication with distal ablation probe 24, such as first and second lumens 74 and 76, described hereinbelow with reference to FIGS. 3A-B. For example, the cryogenic fluid (also known as a refrigerant) may comprise nitrogen, nitrous oxide, hydrogen, argon, propane, an alcohol, or carbon dioxide in one of a gas, liquid, critical, or supercritical state, as is known in the art. For example, source 36 of cryogenic fluid may implement techniques of the cryogenic systems described in U.S. Pat. No. 10,054,262 to Baust et al. and/or U.S. Pat. No. 9,089,316 to Baust et al., both of which are incorporated herein by reference. In case of conflict between definitions provided herein and those provided in these two patents, the definitions provided herein will prevail.

Alternatively or additionally, energy source 35 may comprise a thermal ablation power source, such as a radiofrequency (RF) power source, in which case first and second elongate ablating surfaces 30A and 30B comprise respective sets of one or more RF ablation electrodes (not shown, but similar to sensing electrodes 120 described hereinbelow with reference to FIG. 4C). In this case, ablation system 10 may use RF ablation instead of, or in addition to, cryogenic ablation.

Further alternatively or additionally, energy source 35 comprises a non-thermal energy source, such as pulsed field ablation energy source, as is known in the art.

Typically, ablation system 10 further comprises an operator handle, as is known in the art, and, optionally, an umbilical connector, as is known in the art.

For some applications, distal ablation probe 24, when unconstrained, has a greatest dimension D1 of at least 4 cm, no more than 10 cm, and/or between 4 and 10 cm.

For some applications, distal ablation probe 24, when unconstrained, has a greatest major dimension D2 and a greatest minor dimension D3 perpendicular to each other. For some applications, the dimensions have one or more of the following absolute or relative values:
the greatest major dimension D2 is at least 3 times the greatest minor dimension D3, such as at least 4 times the greatest minor dimension,
the greatest major dimension D2 is at least 4 cm, no more than 10 cm, and/or between 4 and 10 cm, and/or
the greatest minor dimension D3 is at least 0.5 cm, no more than 2 cm, and/or between 0.5 and 2 cm.

For applications in which distal ablation probe 24 comprises elongate distal shaft 32, all of the above-mentioned dimensions are defined by an outer perimeter of elongate distal shaft 32 that defines an outer border of distal ablation probe 24.

For some applications in which distal ablation probe 24 comprises elongate distal shaft 32, elongate distal shaft 32 has a length, measured along an axis thereof, of at least 4 cm, no more than 8 cm, and/or between 4 and 8 cm (because the length is measured along the axis, the length remains the same when distal ablation probe 24 is unconstrained and when elongate distal shaft 32 is constrained to a straight delivery configuration).

For some applications, elongate proximal shaft 22 has a length of at least 80 cm (e.g., at least 100 cm), no more than 150 cm (e.g., no more than 130 cm), and/or between 80 cm (e.g., 100 cm) and 150 cm (e.g., 130 cm).

For some applications, first and second elongate ablating surfaces 30A and 30B are coplanar when distal ablation probe 24 is unconstrained, such as shown. Alternatively, they are not coplanar.

For some applications, first and second elongate ablating surfaces 30A and 30B are parallel to each other when distal ablation probe 24 is unconstrained, such as shown in FIGS. 1, 2A-B, and 4D-E. For other applications, first and second elongate ablating surfaces 30A and 30B are not parallel to each other when distal ablation probe 24 is unconstrained.

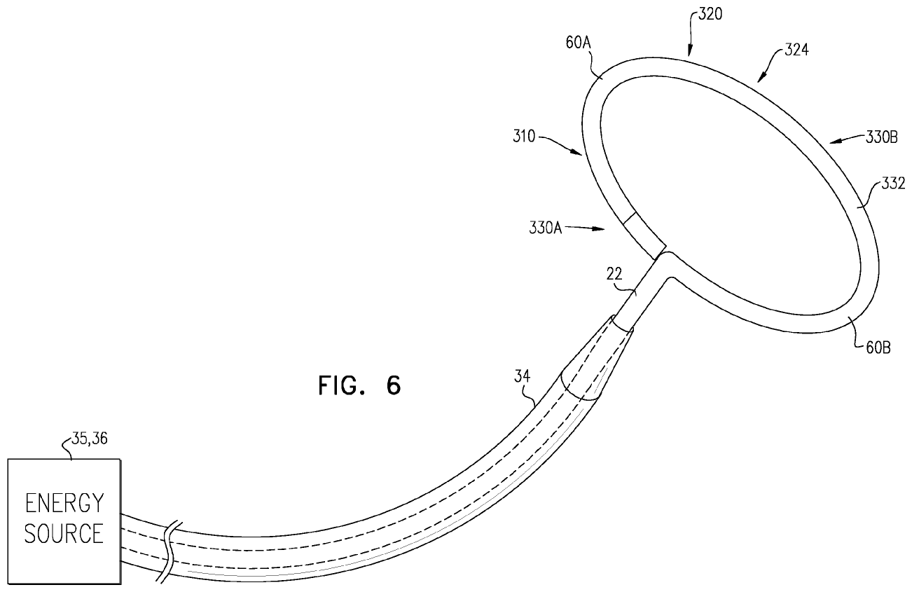

For some applications, first and second elongate ablating surfaces 30A and 30B are straight when distal ablation probe 24 is unconstrained, such as shown in FIGS. 1, 2A-B, and 4D-E. For other applications, first and second elongate ablating surfaces are curved when the distal ablation probe is unconstrained, for example as described hereinbelow with reference to FIG. 6.

For some applications, such as labeled in FIG. 213, when distal ablation probe 24 is unconstrained, a best-fit plane 38 defined by first and second elongate ablating surfaces 30A and 30B forms an angle α (alpha) with a central longitudinal axis 42 of elongate proximal shaft 22 that passes through distal end 26 of elongate proximal shaft 22, the angle between 45 and 90 degrees, such as between 60 and 90 degrees, e.g., between 75 and 90 degrees, such as 90 degrees as shown. For some applications in which distal ablation probe 24 comprises elongate distal shaft 32, a proximal end 44 of elongate distal shaft 32 is supported at distal end 26 of elongate proximal shaft 22, and when distal ablation probe 24 is unconstrained, a proximal portion 46 of elongate distal shaft 32 forms the above-mentioned angle α (alpha) with central longitudinal axis 42 of elongate proximal shaft 22 that passes through distal end 26 of elongate proximal shaft 22.

As used in the present application, including in the claims, a “best-fit plane” of distal ablation probe 24 is the plane that results in the minimum sum of squares of distances between the plane and all points of the volume of distal ablation probe 24. As used in the present application, including in the claims, an angle between two lines or two planes is the smaller of the two supplementary angles between the two lines or two planes, or equals 90 degrees if the two lines or two planes are perpendicular.

For some applications, such as labeled in FIG. 2A, when distal ablation probe 24 is unconstrained, first and second elongate ablating surfaces 30A and 30B run alongside each other for an ablation-surface length L that equals:
at least 4 cm, no more than 8 cm, and/or between 4 and 8 cm, and/or
between 3 and 12 times a closest distance D4 between first and second elongate ablating surfaces 30A and 3013, such as between 4 and 8 times the closest distance D4.

For some applications, closest distance D4 (labeled in FIG. 2A) between first and second elongate ablating surfaces 30A and 30B is at least 5 mm, no more than 20 mm, and/or between 5 and 20 mm when distal ablation probe 24 is unconstrained.

For some applications, a distance between first and second elongate ablating surfaces 30A and 30B does not vary along first and second elongate ablating surfaces 30A and 30B when distal ablation probe 24 is unconstrained, such as shown in FIGS. 1 and 2A-B. For other applications, a distance between first and second elongate ablating surfaces 30A and 30B varies along first and second elongate ablating surfaces 30A and 30B when distal ablation probe 24 is unconstrained, such as in the configuration described hereinbelow with reference to FIG. 6.

For some applications, a distal end 45 of elongate distal shaft 32 is located along first elongate ablating surface 30A when distal ablation probe 24 is unconstrained, such as shown.

As shown in the figures, for some applications, such as in applications in which distal end 45 of elongate distal shaft 32 is located along first elongate ablating surface 30A:
first elongate ablating surface 30A comprises a first elongate discontinuous ablating surface, configured to make the first elongate continuous ablation lesion, and
second elongate ablating surface 30B comprises a second elongate continuous ablating surface, configured to make the second elongate continuous ablation lesion.

First elongate discontinuous ablating surface 30A comprises two elongate ablating surfaces (such as shown) or three or more elongate ablating surfaces (configuration not shown), which together define first elongate discontinuous ablating surface 30A. Despite the small gap between the two or more elongate ablating surfaces, the two or more elongate ablating surfaces together make an elongate continuous ablation segment in the atrial wall, because the ablating surfaces typically ablate tissue up to about 2-3 mm from the ablating surfaces, and thus bridge the small gap between the longitudinally adjacent ablating surfaces.

For other applications, first and second elongate ablating surfaces 30A and 30B comprise first and second elongate continuous ablating surfaces, respectively (configuration not shown).

For still other applications, first and second elongate ablating surfaces 30A and 30B comprise first and second elongate discontinuous ablating surfaces, respectively (configuration not shown).

For some applications, elongate distal shaft 32 is shaped so as to define two curved connecting end portions 60A and 60B that connect first and second elongate ablating surfaces 30A and 30B when distal ablation probe 24 is unconstrained. Typically, the two curved connecting end portions 60A and 60B of the probe also apply ablation energy. Optionally, each of the two curved connecting end portions 60A and 60B has a radius of curvature R (labeled in FIG. 2A) of at least 0.25 cm, no more than 1 cm, and/or between 0.25 and 1 cm.

For some of these applications, distal end 45 of elongate distal shaft 32 physically touches proximal end 44 of elongate distal shaft 32 when distal ablation probe 24 is unconstrained, such as shown in FIGS. 1, 2A-B, and 4D-E. For some of these applications, when distal ablation probe 24 is unconstrained, an inner perimeter 48 of elongate distal shaft 32 surrounds an area 50 of at least 2 cm2, no more than 16 cm2, and/or between 2 and 16 cm2.

For some applications, when distal ablation probe 24 is unconstrained, first elongate ablating surface 30A includes proximal end 44 of elongate distal shaft 32 (which is supported at distal end 26 of elongate proximal shaft 22). Proximal end 44 is located at a location 52 along first elongate ablating surface 30A at a distance D5 from an endpoint 54 of first elongate ablating surface 30A, the distance D5 equal to at least 40%, no more than 60%, and/or between 40% and 60% (e.g., 45% to 55%, e.g., 50%) of the length L of first elongate ablating surface 30A. This location 52 of insertion of elongate proximal shaft 22 into the distal transmissive region may help provide good maneuverability to distal ablation probe 24. This location 52 may also help create a ‘T’ shape, which may have a fixed length (such as shown) or a variable length (for example, the handle of the device may comprise a slider that allows the operator to change a length intraprocedurally; configuration not shown).

Reference is now made to FIGS. 3A and 3B, which are cross-sectional views of elongate distal shaft 32 of distal ablation probe 24 taken along lines IIIA-IIIA and IIIB-IIIB, respectively, of FIG. 2A, in accordance with respective applications of the present invention.

For some applications, first and second elongate ablating surfaces 30A and 30B are configured to apply cryoablation. To this end, for some applications, elongate distal shaft 32 comprises inner and outer tubes 70 and 72. (Outer tube 72 typically defines an outer wall of elongate distal shaft 32.) Inner tube 70 is shaped so as to define a first lumen 74. Inner and outer tubes 70 and 72 together define a second lumen 76 between an outer surface 78 of inner tube 70 and an inner surface 79 of outer tube 72. First and second lumens 74 and 76 are in fluid communication with each other near (e.g., within 2 cm, such as within 1 cm) of distal end 45 of elongate distal shaft 32 of distal ablation probe 24 (and, typically, not elsewhere along elongate distal shaft 32), such as via at least one opening 82 defined through a wall of inner tube 70 (as shown), or via a distal end of inner tube 70 that is recessed proximally from a distal end of outer tube 72 (configuration not shown). First and second lumens 74 and 76 thus together provide a closed-loop system. For some applications, the closed loop system is designed to withstand very high pressures, such as up to approximately 2000 PSI.

Elongate proximal shaft 22 also comprises first and second lumens, which couple first and second lumens 74 and 76 in fluid communication with source 36 of cryogenic fluid, described hereinabove with reference to FIG. 1. For example, such fluid coupling may be arranged such that first lumen 74 delivers the cryogenic fluid and second lumen 76 returns the cryogenic fluid.

For some applications, distal ablation probe 24 comprises a shape memory material 84 (e.g., a Ni—Ti alloy) that causes elongate distal shaft 32 to define first and second elongate ablating surfaces 30A and 30B running alongside each other when elongate distal shaft 32 is unconstrained. For example, distal ablation probe 24 may comprise a spine 86, which comprises shape memory material 84. Optionally, spine 86 extends proximally through elongate proximal shaft 22 to the operator, to improve rigidity of elongate proximal shaft 22. For some applications, shape memory material 84 becomes more flexible (e.g., floppy) at very low temperatures, which may allow distal ablation probe 24 to comply with non-flat cardiac wall surfaces; as distal ablation probe 24 becomes colder, it first freezes to the cardiac wall, and remains frozen to the wall as the probe becomes floppier at lower, cryogenic temperatures.

Typically, elongate distal shaft 32 comprises a distal plug 92, which maintains the closed-loop system.

Inner and outer tubes 70 and 72 typically comprise one or more biocompatible materials, such as a thermoplastic elastomer, e.g., PEBA.

Although distal ablation probe 24 is shown as comprising only first and second elongate ablating surfaces 30A and 30B, for some applications, the distal ablation probe further comprises one or more additional elongate ablating surfaces, such as a total of three or four elongate ablating surfaces.

Reference is now made to FIGS. 4A-F, which are schematic illustrations of a method for treating atrial arrhythmia (e.g., complex atrial arrhythmia), such as atrial fibrillation or atrial flutter, in accordance with an application of the present invention.

The method comprises advancing (typically transvascularly, such as transvenously advancing), in a transcatheter procedure, into an atrium 100 of a heart, distal ablation probe 24 that is supported at distal end 26 of elongate proximal shaft 22 of ablation catheter 20, such as shown in FIGS. 4A-B. Typically, during the advancing, distal ablation probe 24 is constrained within intravascular delivery sheath 34, optionally such that elongate distal shaft 32 is constrained by intravascular delivery sheath 34, such that first and second elongate ablating surfaces 30A and 30B are disposed at respective, non-longitudinally-overlapping locations along the intravascular delivery sheath. Typically, the atrium is a left atrium 102 (such as shown), and distal ablation probe 24 is first advanced into a right atrium 104, using techniques known in the art, such as shown in FIG. 4A, and is then advanced through an interatrial septum 106 (e.g., at the fossa ovalis) to left atrium 102, using techniques known in the art, such as shown in FIG. 4B. Alternatively, the atrium is a right atrium (configuration not shown).

As shown in FIGS. 4C-D, distal ablation probe 24 is deployed in atrium 100 (e.g., left atrium 102) such that distal ablation probe 24 is shaped so as to define first and second elongate ablating surfaces 30A and 30B running alongside each other.

As shown in FIGS. 4E-F, using distal ablation probe 24, one or more ablation lesions 108 (i.e., regions of cell and/or tissue death) are made in an atrial wall 112. The one or more ablation lesions 108 include at least first and second elongate continuous ablation lesion segments 110A and 110B. First and second elongate ablating surfaces 30A and 30B make first and second elongate continuous ablation lesion segments 110A and 110B, respectively.

The locations of elongate continuous ablation lesion segments 111A and 111B are selected to connect electrically inert boundaries of the atrial wall (typically, veins and/or valves). For example, elongate continuous ablation lesion segments 110A and 110B may generally extend between:
an orifice 112A of a left superior pulmonary vein (LSPV) and an orifice 112B of a right superior pulmonary vein (RSPV) (such as shown),
an RSPV and a mitral valve (MV) (sometimes also known in the art as an anterior oblique or “seatbelt” lesion) (configuration not shown),
a right inferior pulmonary vein (RIPV) and an MV (configuration not shown),
a left inferior pulmonary vein (LIPV) and an RIPV (configuration not shown),
an LSPV and an RIPV (configuration not shown), or
an RSPV and an LIPV (configuration not shown).

In configurations in which distal ablation probe 24 is T-shaped, the nature of the T shape provides the device with the versatility to make many different desired ablation lines.

For some applications, such as shown, distal ablation probe 24 is configured to make a single ablation lesion 108 that includes at least first and second continuous ablation lesion segments 110A and 110B. In these applications, ablation lesion 108 additionally includes additional lesion segments that join together first and second elongate continuous ablation lesion segments 110A and 110B, such as two curved end ablation lesion segments 114A and 114B, which are made by the two curved connecting end portions 60A and 60B.

For other applications, distal ablation probe 24 is configured to make a plurality of separate ablation lesions 108 that include at least first and second elongate continuous ablation lesion segments 110A and 110B, for example, two separate ablation lesions that include at least first and second elongate continuous lesion segments 110A and 110B, respectively (configuration not shown).

Typically, distal ablation probe 24 is not used for performing pulmonary vein isolation. Optionally, pulmonary vein isolation, such as conventional pulmonary vein isolation, is performed in combination with the ablation described herein, typically using one or more ablation probes separate from distal ablation probe 24, as is known in the art. Alternatively, in some applications, the distal ablation probe is also used for performing pulmonary vein isolation after or before performing the linear ablation techniques described herein. In these applications, the distal ablation probe is configured to be transitionable between the configuration described herein (having at least two spaced-apart first and second elongate ablating surfaces running alongside each other) and an elliptical configuration which is shaped to surround and isolate openings of two pulmonary veins at the same time. For example, to provide control of the transition, control wires may be provided that are slidable through the elongate proximal shaft from an external control handle.

For some applications, such as schematically illustrated in FIG. 4E, distal ablation probe 24 comprises one or more sensing electrodes 120, which are used to sense electrical activity in the cardiac wall to ascertain whether the elongate continuous ablation lesion segments 110A and 110B have been properly made. (For clarity of illustration, the one or more sensing electrodes 120 are shown in FIG. 4E but not in the other figures; in practice, one or more sensing electrodes 120 would be present or not present during all stages of the deployment.)

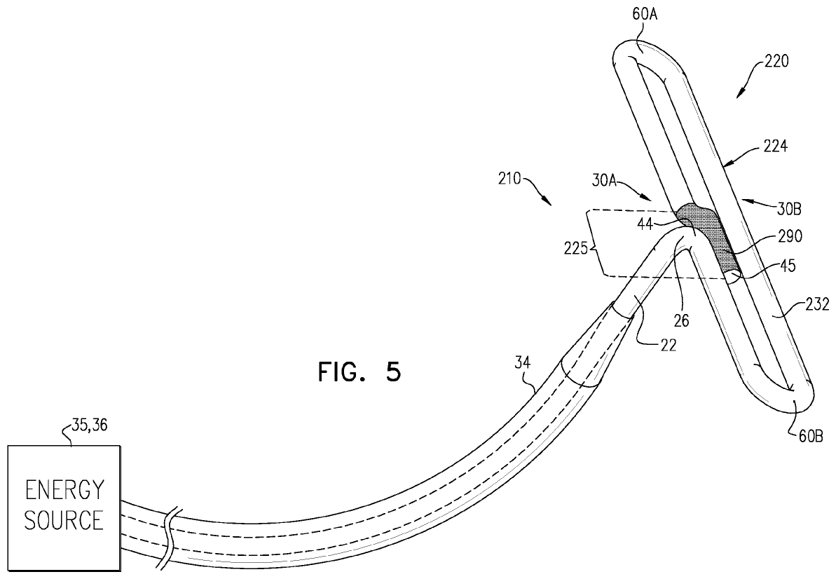

Reference is now made to FIG. 5, which is a schematic illustration of an ablation system 210 for treating atrial arrhythmia, in accordance with an application of the present invention. Other than as described below, ablation system 210 is identical to ablation system 10, described hereinabove with reference to FIGS. 1-4F, and may implement any of the features thereof, mutatis mutandis. Ablation system 210 comprises an ablation catheter 220, which comprises a distal ablation probe 224, which comprises an elongate distal shaft 232. Other than as described below, ablation catheter 220, distal ablation probe 224, and elongate distal shaft 232 are identical to ablation catheter 20, distal ablation probe 24, and elongate distal shaft 32, respectively, described hereinabove with reference to FIGS. 1-4F, and may implement any of the features thereof, mutatis mutandis.

In this configuration, elongate distal shaft 232 comprises a flexible (e.g., floppy) distal tip 290, which is configured to increase the safety of the device when being introduced into and advanced within the atrium. Flexible distal tip 290 is more flexible than a more proximal portion of elongate distal shaft 232, in order to reduce potential endocardial trauma. For example, the more proximal portion of elongate distal shaft 232, unlike flexible distal tip 290, may comprise a reinforcement coil (e.g., comprising a metal, such as a Ni—Ti alloy) to provide increased burst strength. Optionally, both flexible distal tip 290 and the more proximal portion of elongate distal shaft 232 comprise a braid (e.g., a metal braid, such as a stainless steel braid) to provide increased tensile strength). For example, flexible distal tip 290 may have a length of between 0.5 and 2 cm.

Flexible distal tip 290 is typically configured not to apply ablation. Thus, for configurations in which elongate distal shaft 232 comprises first and second lumens 74 and 76, such as described hereinabove with reference to FIGS. 3A and 3B, the first and the second lumens typically do not extend distally to within flexible distal tip 290.

For some of these applications, when distal ablation probe 24 is unconstrained, a distal end portion of elongate distal shaft 232 and a proximal end portion 225 of elongate distal shaft 232 physically touch and run alongside each other, e.g., for a distance of at least 5 mm, no more than 40 mm, and/or between 5 and 40 mm.

For applications in which elongate distal shaft 232 comprises distal plug 92, such as described hereinabove with reference to FIGS. 3A and 3B, flexible distal tip 290 typically extends distally from distal plug 92.

Reference is now made to FIG. 6, which is a schematic illustration of an ablation system 310 for treating atrial arrhythmia, in accordance with an application of the present invention. Other than as described below, ablation system 310 is identical to ablation system 10, described hereinabove with reference to FIGS. 1-4F, and may implement any of the features thereof, mutatis mutandis. Ablation system 310 comprises an ablation catheter 320, which comprises a distal ablation probe 324, which comprises an elongate distal shaft 332. Other than as described below, ablation catheter 320, distal ablation probe 324, and elongate distal shaft 332 are identical to ablation catheter 20, distal ablation probe 24, and elongate distal shaft 32, respectively, described hereinabove with reference to FIGS. 1-4F, and may implement any of the features thereof, mutatis mutandis. Alternatively or additionally, ablation system 310 may also implement any of the features of ablation system 210, described hereinabove with reference to FIG. 5.

In this configuration, distal ablation probe 324 is shaped, when unconstrained, so as to define at least first and second elongate ablating surfaces 330A and 330B running alongside each other. First and second elongate ablating surfaces 330A and 330B are curved when distal ablation probe 324 is unconstrained. For example, the first and the second elongate ablating surfaces have respective radii of curvature, each of which is at least 0.2 cm, no more than 1.2 cm, and/or between 0.2 and 1.2 cm.

A distance between first and second elongate ablating surfaces 330A and 330B varies along first and second elongate ablating surfaces 330A and 330B when distal ablation probe 324 is unconstrained.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An ablation catheter for treating atrial arrhythmia, comprising:
an elongate proximal shaft; and
a distal ablation probe, which is (a) supported at a distal end of the elongate proximal shaft, (b) shaped, when unconstrained, so as to define at least first and second elongate ablating surfaces running alongside each other, and (c) configured to make, in an atrial wall of a heart, one or more ablation lesions that include at least first and second elongate continuous ablation lesion segments that are spaced apart and run alongside each other.

2. The ablation catheter according to claim 1, wherein the first and the second elongate ablating surfaces comprise first and second elongate continuous ablating surfaces, respectively.

3. The ablation catheter according to claim 1,
wherein the first elongate ablating surface comprises a first elongate discontinuous ablating surface, configured to make the first elongate continuous ablation lesion, and
wherein the second elongate ablating surface comprises a second elongate continuous ablating surface, configured to make the second elongate continuous ablation lesion.

4. The ablation catheter according to claim 1, wherein the first and the second elongate ablating surfaces comprise first and second elongate discontinuous ablating surfaces, respectively.

5. The ablation catheter according to claim 1, wherein the distal ablation probe, when unconstrained, has greatest major and minor dimensions perpendicular to each other, the greatest major dimension at least 3 times the greatest minor dimension.

6. The ablation catheter according to claim 5, wherein the greatest major dimension is at least 4 times the greatest minor dimension.

7. The ablation catheter according to claim 1, wherein the distal ablation probe, when unconstrained, has a greatest dimension of between 4 and 10 cm.

8. The ablation catheter according to claim 1, wherein the first and the second elongate ablating surfaces are coplanar when the distal ablation probe is unconstrained.

9. The ablation catheter according to claim 1, wherein the first and the second elongate ablating surfaces are parallel to each other when the distal ablation probe is unconstrained.

10. The ablation catheter according to claim 1, wherein the first and the second elongate ablating surfaces run alongside each other for an ablation-surface length of between 4 and 8 cm when the distal ablation probe is unconstrained.

11. The ablation catheter according to claim 1, wherein a closest distance between the first and the second elongate ablating surfaces is between 5 and 20 mm when the distal ablation probe is unconstrained.

12. The ablation catheter according to claim 1, wherein the distal ablation probe comprises one or more sensing electrodes.

13. The ablation catheter according to any one of claims 1-12, wherein a distance between the first and the second elongate ablating surfaces does not vary along the first and the second elongate ablating surfaces when the distal ablation probe is unconstrained.

14. The ablation catheter according to any one of claims 1-12, wherein a distance between the first and the second elongate ablating surfaces varies along the first and the second elongate ablating surfaces when the distal ablation probe is unconstrained.

15. The ablation catheter according to any one of claims 1-12, wherein the first and the second elongate ablating surfaces are straight when the distal ablation probe is unconstrained.

16. The ablation catheter according to any one of claims 1-12, wherein the first and the second elongate ablating surfaces are curved when the distal ablation probe is unconstrained.

17. The ablation catheter according to claim 16, wherein, when the distal ablation probe is unconstrained, the first and the second elongate ablating surfaces have respective radii of curvature, each of which is between 0.2 and 1.2 cm.

18. The ablation catheter according to any one of claims 1-12, wherein the distal ablation probe comprises an elongate distal shaft that is shaped so as to define the first and the second elongate ablating surfaces when the distal ablation probe is unconstrained.

19. The ablation catheter according to claim 18, wherein, when the distal ablation probe is unconstrained, the elongate distal shaft has greatest major and minor dimensions perpendicular to each other, and wherein the greatest major dimension equals at least 3 times the greatest minor dimension.

20. The ablation catheter according to claim 19, wherein the greatest major dimension is at least 4 times the greatest minor dimension.

21. The ablation catheter according to claim 18,
wherein a proximal end of the elongate distal shaft is supported at the distal end of the elongate proximal shaft, and
wherein, when the distal ablation probe is unconstrained, a proximal portion of the elongate distal shaft forms an angle with a central longitudinal axis of the elongate proximal shaft that passes through the distal end of the elongate proximal shaft, the angle between 45 and 90 degrees.

22. The ablation catheter according to claim 21, wherein the angle is between 60 and 90 degrees.

23. The ablation catheter according to claim 18,
wherein the first elongate ablating surface comprises a first elongate discontinuous ablating surface, configured to make the first elongate continuous ablation lesion, and
wherein the second elongate ablating surface comprises a second elongate continuous ablating surface, configured to make the second elongate continuous ablation lesion.

24. The ablation catheter according to claim 23, wherein a distal end of the elongate distal shaft is located along the first elongate ablating surface when the distal ablation probe is unconstrained.

25. The ablation catheter according to claim 24, wherein a distal end of the elongate distal shaft physically touches a proximal end of the elongate distal shaft when the distal ablation probe is unconstrained.

26. The ablation catheter according to claim 25, wherein, when the distal ablation probe is unconstrained, an inner perimeter of the elongate distal shaft surrounds an area of between 2 and 16 cm2.

27. The ablation catheter according to claim 18, wherein the elongate distal shaft is shaped so as to define two curved connecting end portions that connect the first and the second elongate ablating surfaces when the distal ablation probe is unconstrained.

28. The ablation catheter according to claim 18, wherein the first and the second elongate ablating surfaces are curved and the elongate distal shaft is ovaloid when the distal ablation probe is unconstrained.

29. The ablation catheter according to claim 18, wherein the elongate distal shaft is stadium-shaped when the distal ablation probe is unconstrained.

30. An ablation system comprising the ablation catheter according to claim 18, the ablation system further comprising an intravascular delivery sheath, in which the ablation catheter is removably disposed for delivery such that the elongate distal shaft is constrained by the intravascular delivery sheath, such that the first and the second elongate ablating surfaces are disposed at respective, non-longitudinally-overlapping locations along the intravascular delivery sheath.

31. The ablation catheter according to claim 18, wherein the distal ablation probe comprises a shape memory material that causes the elongate distal shaft to define the first and the second ablating surfaces running alongside each other when the elongate distal shaft is unconstrained.

32. The ablation catheter according to claim 18, wherein, when the distal ablation probe is unconstrained:
a proximal end of the elongate distal shaft is supported at the distal end of the elongate proximal shaft,
the first elongate ablating surfaces includes the proximal end of the elongate distal shaft,
the proximal end is located at a location along the first elongate ablating surface at a distance from an endpoint of the first elongate ablating surface, the distance equal to between 40% and 60% of a length of the first elongate ablating surface.

33. The ablation catheter according to any one of claims 1-12, wherein when the distal ablation probe is unconstrained, a best-fit plane defined by the first and the second elongate ablating surfaces forms an angle with a central longitudinal axis of the elongate proximal shaft that passes through the distal end of the elongate proximal shaft, the angle between 45 and 90 degrees.

34. The ablation catheter according to claim 33, wherein the angle is between 60 and 90 degrees.

35. The ablation catheter according to any one of claims 1-12, wherein the first and the second elongate ablating surfaces are configured to apply cryoablation.

36. The ablation catheter according to claim 35,
wherein the distal ablation probe comprises an elongate distal shaft that is shaped so as to define the first and the second elongate ablating surfaces when the distal ablation probe is unconstrained
wherein the distal elongate shaft comprises inner and outer tubes,
wherein the inner tube is shaped so as to define a first lumen,
wherein the inner and the outer tubes together define a second lumen between an outer surface of the inner tube and an inner surface of the outer tube, and
wherein the first and the second lumens are in fluid communication with each other near a distal end of the distal elongate shaft.

37. An ablation system comprising the ablation catheter according to claim 36, the ablation system further comprising a source of cryogenic fluid coupled in fluid communication with the first and the second lumens.

38. The ablation catheter according to any one of claims 1-12, wherein the first and the second elongate ablating surfaces comprise respective sets of one or more ablation electrodes.

39. A method for treating atrial arrhythmia comprising:
advancing, in a transcatheter procedure, into an atrium of a heart, a distal ablation probe that is supported at a distal end of an elongate proximal shaft of an ablation catheter;
deploying the distal ablation probe in the atrium such that the distal ablation probe is shaped so as to define at least first and second elongate ablating surfaces running alongside each other; and
using the distal ablation probe, making, in an atrial wall, one or more ablation lesions that include at least first and second elongate continuous ablation lesion segments that are spaced apart and run alongside each other.

40. The method according to claim 39, wherein making the one or more ablation lesions comprises making the first and the second elongate continuous ablation lesion segments at respective locations in the atrial wall that connect electrically inert boundaries of the atrial wall.

41. The method according to claim 40, wherein making the first and the second elongate continuous ablation lesion segments comprises making the first and the second elongate continuous ablation lesion segments generally extending between:
an orifice of a left superior pulmonary vein (LSPV) and an orifice of a right superior pulmonary vein (RSPV),
an RSPV and a mitral valve (MV),
a right inferior pulmonary vein (RIPV) and an MV,
a left inferior pulmonary vein (LIPV) and an RIPV,
an LSPV and an RIPV, or
an RSPV and an LIPV.

42. The method according to claim 39, wherein the method does not comprise using the distal ablation probe to perform pulmonary vein isolation.

43. The method according to claim 39, wherein advancing the distal ablation probe while the ablation catheter is removably disposed for delivery in an intravascular delivery sheath, such that the elongate distal shaft is constrained by the intravascular delivery sheath, such that the first and the second elongate ablating surfaces are disposed at respective, non-longitudinally-overlapping locations along the intravascular delivery sheath.

44. The method according to claim 39, wherein the first and the second elongate ablating surfaces include first and second elongate continuous ablating surfaces, respectively.

45. The method according to claim 39,
wherein the first elongate ablating surface includes a first elongate discontinuous ablating surface, configured to make the first elongate continuous ablation lesion, and
wherein the second elongate ablating surface includes a second elongate continuous ablating surface, configured to make the second elongate continuous ablation lesion.

46. The method according to claim 39, wherein the first and the second elongate ablating surfaces include first and second elongate discontinuous ablating surfaces, respectively.

47. The method according to claim 39, wherein the distal ablation probe, when unconstrained, has greatest major and minor dimensions perpendicular to each other, the greatest major dimension at least 3 times the greatest minor dimension.

48. The method according to claim 47, wherein the greatest major dimension is at least 4 times the greatest minor dimension.

49. The method according to claim 39, wherein the distal ablation probe, when unconstrained, has a greatest dimension of between 4 and 10 cm.

50. The method according to claim 39, wherein the first and the second elongate ablating surfaces are coplanar when the distal ablation probe is unconstrained.

51. The method according to claim 39, wherein the first and the second elongate ablating surfaces are parallel to each other when the distal ablation probe is unconstrained.

52. The method according to claim 39, wherein the first and the second elongate ablating surfaces are straight when the distal ablation probe is unconstrained.

53. The method according to claim 39, wherein the first and the second elongate ablating surfaces are curved when the distal ablation probe is unconstrained.

54. The method according to claim 53, wherein, when the distal ablation probe is unconstrained, the first and the second elongate ablating surfaces have respective radii of curvature, each of which is between 0.2 and 1.2 cm.

55. The method according to claim 39, wherein the distal ablation probe includes an elongate distal shaft that is shaped so as to define the first and the second elongate ablating surfaces when the distal ablation probe is unconstrained.

56. The method according to claim 55, wherein, when the distal ablation probe is unconstrained, the elongate distal shaft has greatest major and minor dimensions perpendicular to each other, and wherein the greatest major dimension equals at least 3 times the greatest minor dimension.

57. The method according to claim 56, wherein the greatest major dimension is at least 4 times the greatest minor dimension.

58. The method according to claim 55,
wherein a proximal end of the elongate distal shaft is supported at the distal end of the elongate proximal shaft, and
wherein, when the distal ablation probe is unconstrained, a proximal portion of the elongate distal shaft forms an angle with a central longitudinal axis of the elongate proximal shaft that passes through the distal end of the elongate proximal shaft, the angle between 45 and 90 degrees.

59. The method according to claim 58, wherein the angle is between 60 and 90 degrees.

60. The method according to claim 55,
wherein the first elongate ablating surface includes a first elongate discontinuous ablating surface, configured to make the first elongate continuous ablation lesion, and
wherein the second elongate ablating surface includes a second elongate continuous ablating surface, configured to make the second elongate continuous ablation lesion.

61. The method according to claim 60, wherein a distal end of the elongate distal shaft is located along the first elongate ablating surface when the distal ablation probe is unconstrained.

62. The method according to claim 61, wherein a distal end of the elongate distal shaft physically touches a proximal end of the elongate distal shaft when the distal ablation probe is unconstrained.

63. The method according to claim 62, wherein, when the distal ablation probe is unconstrained, an inner perimeter of the elongate distal shaft surrounds an area of between 2 and 16 cm2.

64. The method according to claim 55, wherein the elongate distal shaft is shaped so as to define two curved connecting end portions that connect the first and the second elongate ablating surfaces when the distal ablation probe is unconstrained.

65. The method according to claim 55, wherein the first and the second elongate ablating surfaces are curved and the elongate distal shaft is ovaloid when the distal ablation probe is unconstrained.

66. The method according to claim 55, wherein the elongate distal shaft is stadium-shaped when the distal ablation probe is unconstrained.

67. The method according to claim 55, wherein the distal ablation probe includes a shape memory material that causes the elongate distal shaft to define the first and the second ablating surfaces running alongside each other when the elongate distal shaft is unconstrained.

68. The method according to claim 55, wherein, when the distal ablation probe is unconstrained:
a proximal end of the elongate distal shaft is supported at the distal end of the elongate proximal shaft,
the first elongate ablating surfaces includes the proximal end of the elongate distal shaft, the proximal end is located at a location along the first elongate ablating surface at a distance from an endpoint of the first elongate ablating surface, the distance equal to between 40% and 60% of a length of the first elongate ablating surface.

69. The method according to claim 39, wherein when the distal ablation probe is unconstrained, a best-fit plane defined by the first and the second elongate ablating surfaces forms an angle with a central longitudinal axis of the elongate proximal shaft that passes through the distal end of the elongate proximal shaft, the angle between 45 and 90 degrees.

70. The method according to claim 69, wherein the angle is between 60 and 90 degrees.

71. The method according to claim 39, wherein the first and the second elongate ablating surfaces run alongside each other for an ablation-surface length of between 4 and 8 cm when the distal ablation probe is unconstrained.

72. The method according to claim 39, wherein a closest distance between the first and the second elongate ablating surfaces is between 5 and 20 mm when the distal ablation probe is unconstrained.

73. The method according to claim 39, wherein a distance between the first and the second elongate ablating surfaces does not vary along the first and the second elongate ablating surfaces when the distal ablation probe is unconstrained.

74. The method according to claim 39, wherein a distance between the first and the second elongate ablating surfaces varies along the first and the second elongate ablating surfaces when the distal ablation probe is unconstrained.

75. The method according to claim 39, wherein the first and the second elongate ablating surfaces are configured to apply cryoablation.

76. The method according to claim 75,
wherein the distal ablation probe includes an elongate distal shaft that is shaped so as to define the first and the second elongate ablating surfaces when the distal ablation probe is unconstrained
wherein the distal elongate shaft includes inner and outer tubes,
wherein the inner tube is shaped so as to define a first lumen,
wherein the inner and the outer tubes together define a second lumen between an outer surface of the inner tube and an inner surface of the outer tube, and
wherein the first and the second lumens are in fluid communication with each other near a distal end of the distal elongate shaft.

77. The method according to claim 76, further comprising coupling a source of cryogenic fluid in fluid communication with the first and the second lumens.

78. The method according to claim 39, wherein the first and the second elongate ablating surfaces include respective sets of one or more ablation electrodes.

79. The method according to claim 39, wherein the distal ablation probe includes one or more sensing electrodes.

80. A system according to claim 1, in combination with any one or more of the other claims.

81. A method according to claim 39, in combination with any one or more of the other claims.
